(19) United States
(12) Patent Application Publication
(10) Pub. No.: US 2021/0015629 A1
(43) Pub. Date: Jan. 21, 2021

(54) Expandable TLIF or PLIF Implants

(71) Applicants: Judson Cook, Austin, TX (US); Thomas Zink, San Antonio, TX (US)

(72) Inventors: Judson Cook, Austin, TX (US); Thomas Zink, San Antonio, TX (US)

(21) Appl. No.: 16/929,516

(22) Filed: Jul. 15, 2020

Related U.S. Application Data
(60) Provisional application No. 62/874,171, filed on Jul. 15, 2019.

(51) Int. Cl. A61F 2/44; A61F 2/46

(52) U.S. Cl. CPC A61F 2/4455; A61F 2002/30985; A61F 2/4611

(57) ABSTRACT

A rotatable and/or expandable PLIF or TLIF implant that promotes bone growth and that is capable of expanding medial-laterally to achieve a larger overall footprint for greater stability of the construct. The implant comprises a body with substantially parallel posterior and anterior sidewalls, and a pair of superior and inferior faces. The implant also comprises at least one depression or opening in the anterior or posterior sidewalls for engagement by an insertion tool, and at least one chamfer on the superior and inferior faces to allow for easier insertion.

### CROSS-REFERENCE TO RELATED APPLICATION

The present application claims priority to, and the benefit of, U.S. Provisional Application No. 62/874,171, which was filed on Jul. 15, 2019 and is incorporated herein by reference in its entirety.

### FIELD OF THE INVENTION

The present invention relates generally to a vertebral implant, and more particularly to additively manufactured transforaminal lumbar interbody fusion (TLIF) implants and posterior lumbar interbody fusion (PLIF) implants that can be rotated about their longitudinal axis once in the disc space to increase the amount of distraction, and/or expanded at a front or forward end of the implant to achieve a larger footprint on the endplate. Notwithstanding, the present invention contemplates both a pivotally expanding implant device and a parallel expanding implant device.

### BACKGROUND

A number of medical conditions can cause severe back pain including, without limitation, compression of the spinal cord nerve roots, degenerative disc disease, tumors, and trauma. Intervertebral fusion is one surgical method of alleviating back pain. In an intervertebral fusion surgical procedure, two adjacent vertebral bodies are fused together by removing the affected intervertebral disc, and inserting posteriorly an implant that would allow for bone to grow between the two adjacent vertebral bodies to bridge the gap (oftentimes referred to as the disc space) created by the intervertebral disc removal.

A number of different implant materials and implant designs have historically been used for intervertebral/interbody fusion and for vertebral body replacement with varying degrees of success. Current implant materials used include metals, radiolucent materials including plastics, elastic and polymeric materials, ceramic, and allografts, and current implant designs vary from threaded cylindrical implants to generally rectangular cages having teeth-like protrusions.

Additionally, for patients that require intervertebral/interbody fusion surgery to treat such ailments as degenerative disc disease, deformity and instability, the PLIF cage has been shown to be somewhat effective. However, there remains a number of limitations associated with the use of the PLIF impaction design for such applications. By way of example and not limitation, the force needed to hammer in such an implant can damage the patient's vertebral bodies and otherwise cause unnecessary trauma. Additionally, most PLIF devices are inserted sideways with a manual tool that grips the exterior surface of the PLIF device, and then rotates the same within the disc space. However, because of the relatively weak sidewalls of existing PLIF devices, and the positioning of the manual tool on the exterior surface of the sidewalls of the PLIF device and the forces applied thereon, the PLIF devices tend to either break or become deformed during the insertion procedure, or are incapable of exerting enough force on the adjacent vertebrae to achieve successful insertion of the PLIF device and/or the desired amount of distraction. Furthermore, the existing PLIF devices generally only expand in one plane, namely the vertical plane. Therefore, the existing PLIF devices do not achieve a large enough overall footprint to provide greater stability, and are therefore an ineffective solution.

Therefore, there is a long felt need in the art for a rotatable and/or expandable PLIF implant that can be inserted into the disc space with less force and less trauma to the patient's vertebral bodies, and without bending, breaking or otherwise causing damage to the PLIF implant and/or the insertion tool. There is also a long felt need in the art for a rotatable and/or expandable PLIF implant that promotes bone growth and that is also capable of expanding in the horizontal plane (as well as the vertical plane) to achieve a larger overall footprint for greater stability of the construct. Additionally, there is a long felt need in art for PLIF implants that can be expanded pivotally or parallelly. Finally, there is a long felt need in the art for an improved PLIF implant that is relatively easy to manufacture and use.

While this specification makes specific reference to a posterior lumbar interbody fusion (PLIF) application of the present invention, it will be appreciated by those of ordinary skill in the art that aspects of the present invention are also equally amenable to other like applications, such as a transforaminal lumbar interbody fusion (TLIF) application, etc.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The present invention includes a PLIF or TLIF implant comprising a body with substantially parallel posterior and anterior sidewalls, and a pair of superior and inferior faces. The PLIF or TLIF implant also comprises at least one depression or opening in the anterior or posterior sidewalls for engagement by an insertion tool, and at least one chamfer on the superior and inferior faces to allow for easier insertion into the patient. The superior and inferior faces also preferably comprise a rounded edge along a perimeter of the faces. The chamfer on the superior and inferior faces facilitates easier insertion of the implant in either a straight or oblique posterior approach, so that a single implant can be used to provide balanced support to the spinal column.

Furthermore, the PLIF implant of the present invention is capable of expanding medial-laterally in vivo. More specifically, the PLIF implant can expand via a parallel expansion design which causes the PLIF implant to open medial-laterally, or via a pivot expansion design which only expands on the anterior side of the implant. Once expanded, flowable bone graft can be inserted or pushed into the voids created by the expansion of the PLIF or TLIF implant to promote bone growth (or osteo-integration). Further, the PLIF and TLIF implants of the present invention may be manufactured with webbing or other honey-combed like structure or mesh on the lateral sections of the sidewalls to promote further bone growth and osteo-integration of the implant after implantation.

In another embodiment of the present invention, the PLIF implant may be inserted via an insertion tool within the depression or slots that exist between the sidewalls of the PLIF implant. The insertion tool preferably comprises an elongated shaft defining a longitudinal axis and having a proximal end and a distal end. The proximal end comprises a handle and the distal end comprises a spaced apart grip member. The spaced apart grip member further comprises bifurcated shaft portions which securely retain the PLIF implant therebetween during the implantation procedure.

More specifically, the bifurcated shaft portions of the insertion tool are inserted into the slots formed between the sidewalls of the PLIF or TLIF implant, and, at the same time, a threaded portion of the insertion tool is inserted into an opening, which secures the implant to the insertion tool, and reduces the likelihood that the implant or insertion tool will bend or break during the implantation procedure, thereby reducing risk to the patient. Once the implant has been inserted longitudinally into the disc space, the implant may be rotated approximately 90 degrees within the disc space, and can be rotated in the clockwise or counterclockwise direction to suit user need and/or preference. Because the PLIF or TLIF implant is typically taller than it is wide, rotating the implant after implantation in the disc space can increase the amount of distraction or space between the patient's adjacent vertebras. Nonetheless, the implant can also be positioned in a straight PLIF configuration as well (i.e., not rotated after implantation), depending on the wants and needs of the surgeon performing the procedure and/or the particular patient requirements.

Once inserted and rotated, the insertion tool may then be used to expand the implant medial-laterally. Specifically, the insertion tool mechanically separates the implant either by pushing the implant open medial-laterally; or pivoting the implant open on its anterior side. Once the implant has been expanded (i.e., medial laterally or pivotally), flowable graft material can be inserted or otherwise pushed into the voids created by the expansion of the implant to promote bone growth and/or osteo-integration).

Furthermore, in another embodiment of the present invention, two PLIF implants can be placed into the disc space in a direct posterior approach to achieve a larger overall footprint for greater stability of the construct. In use, one or two implants can be used in the disc space depending on the wants and/or needs of the surgeon performing the procedure and/or the particular patient requirements.

In yet another embodiment of the present invention, the PLIF or TLIF implants may be manufactured using additive manufacturing techniques. Additionally, if not manufactured by additive manufacturing techniques, the PLIF or TLIF implants and/or the inserter may be manufactured from titanium, specifically Ti 6 Al 4 V-ELI, or any other suitable material used for installation of the implant in a sterile environment or surgical setting.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1 illustrates a perspective view of one possible embodiment of a parallelly expandable PLIF implant attached to an insertion tool in accordance with the disclosed architecture, and in a closed or unexpanded position.

FIG. 2 illustrates a perspective view of the parallelly expandable PLIF implant of FIG. 1 attached to an insertion tool and expanded medial-laterally in accordance with the disclosed architecture.

FIG. 3 illustrates a perspective view of one possible embodiment of a parallelly expandable PLIF implant having an opening in the rear of the implant and attached to an insertion tool in accordance with the disclosed architecture, and in a closed or unexpanded position.

FIG. 4 illustrates a perspective view of one possible embodiment of a pivotally expandable PLIF implant attached to an insertion tool in accordance with the disclosed architecture, and in a closed or unexpanded position.

FIG. 5 illustrates a perspective view of the pivotally expandable PLIF implant of FIG. 4 attached to an insertion tool and pivotally expanded in accordance with the disclosed architecture.

FIG. 6 illustrates a top perspective view of one possible embodiment of two pivotally expanded PLIF implants positioned within a patient's disc space in accordance with the disclosed architecture.

FIG. 7 illustrates a perspective view of one possible embodiment of two pivotally expanded PLIF implants positioned within a patient's disc space in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof

Generally stated, and in one embodiment thereof, the present invention discloses a rotatable and/or expandable PLIF implant that promotes bone growth and that is capable of expanding, medial laterally or pivotally, in the horizontal plane to achieve a larger overall footprint and to achieve greater stability of the construct. In one embodiment, the PLIF implant comprises a body with substantially parallel posterior and anterior sidewalls, and a pair of superior and inferior faces. The PLIF implant also preferably comprises at least one depression or opening in the anterior or posterior sidewalls for engagement by an insertion tool, and at least one chamfer on the superior and inferior faces to allow for easier insertion of the PLIF implant in the vertebral space. Furthermore, in one embodiment, the PLIF implant expands medial-laterally in vivo. More specifically, the PLIF implant is capable of expanding via a parallel expansion design which pushes the implant open medial-laterally. In an alternative embodiment of the PLIF implant of the present invention, the PLIF implant is pivotally expandable on the anterior side of the PLIF implant. Once expanded, flowable bone graft can be inserted or pushed into the voids created by the expansion to promote bone growth and/or osteo-integration.

Furthermore, it is contemplated that more than one PLIF implant can be inserted into a patient's disc space in a direct posterior approach to achieve a larger overall footprint and provide for greater stability of the construct. More specifically, as explained more fully below and illustrated in the FIGS., two expandable (medially or pivotally) PLIF implants can be inserted into a patient's disc space in a general side by side and spaced apart fashion depending on the wants and/or needs of the surgeon performing the procedure and/or the particular patient requirements.

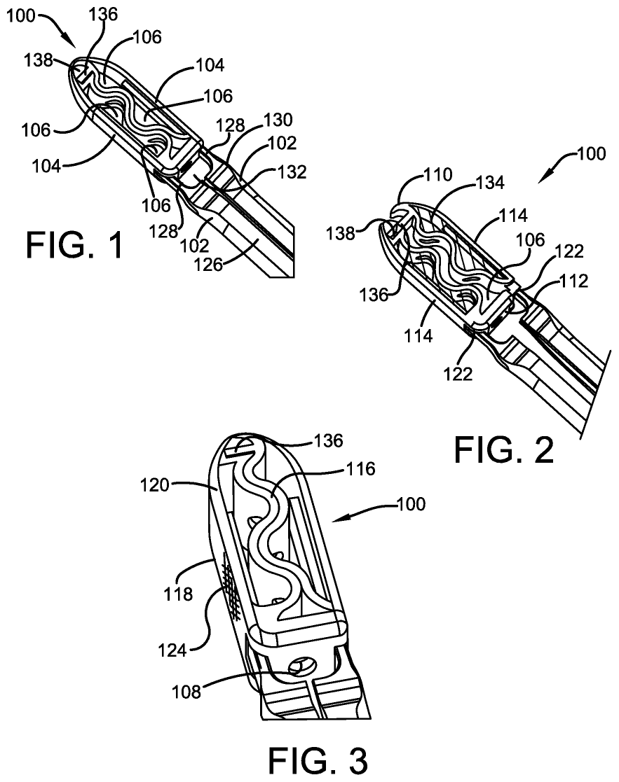

Turning now to the drawings, FIGS. 1-5 disclose a perspective view of various alternative embodiments of a posterior lumbar interbody fusion (PLIF) implant 100. More specifically, FIG. 1 illustrates a perspective view of one possible embodiment of a parallelly expandable PLIF implant 100 attached to an insertion tool 102 in accordance with the disclosed architecture, and in a closed or unexpanded position, and FIG. 2 illustrates a perspective view of the parallelly expandable PLIF implant 100 of FIG. 1 attached to insertion tool 102 and expanded medial-laterally. The PLIF implant 100 is preferably comprised of a body portion 104, one or more graft windows 106 for promoting bone growth and/or osteo-integration, as explained more fully below, and an opening 108 formed therein in the rear of the implant 100 for receipt of a portion of insertion tool 102, as described more fully below. FIG. 3 illustrates a perspective view of one possible embodiment of a parallelly expandable PLIF implant 100 showing the opening 108 in the rear of the implant 100 and attached to an insertion tool 102 in a closed or unexpanded position. The body portion may be further comprised of two halves, wherein the PLIF implant 100 is expandable by repositioning a first half of body portion 104 away from a second half of body portion 104, as best shown in FIG. 2.

Body portion 104 is also preferably comprised of a superior or front end 110, a rear end 112, opposing sides 114, a top 116 and an opposing bottom 118. In a preferred embodiment, front end 110 is generally curved in shape and is comprised of one or more chamfered surfaces 120, preferably tapering off of the top 116 and bottom 118 in the direction of the front end 110. The chamfers 120 on front end 110 facilitate insertion of implant 100 in a generally oblique or straight posterior approach so that a single implant 100 is capable of providing the patient with balanced support to the spinal column once properly implanted.

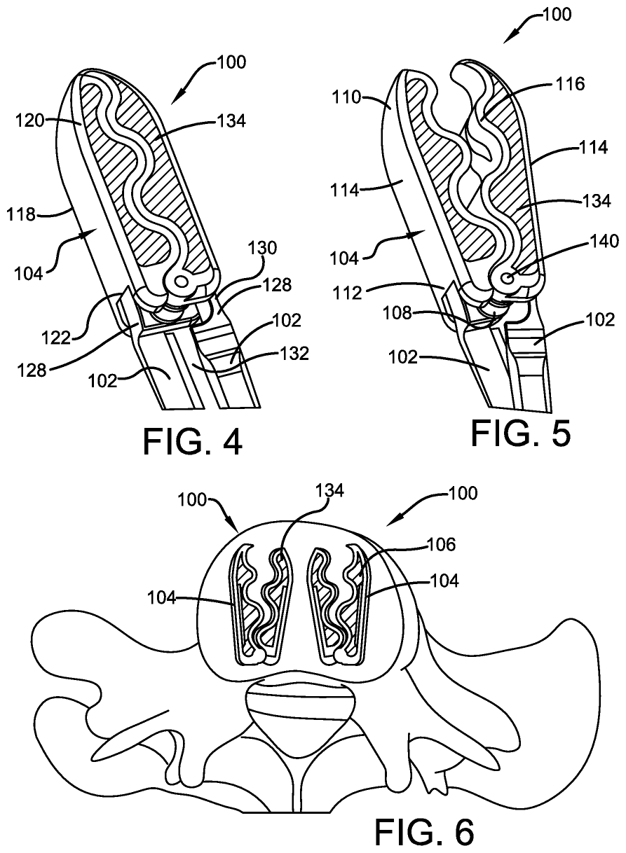

As best shown in FIGS. 2 and 4, rear end 112 of PLIF implant 100 is preferably comprised of two, generally parallel and spaced apart openings 122 formed therein for receipt of a portion of insertion tool 102, which will be explained more fully below. Ideally, the edges of rear end 112 will also be generally rounded or chamfered to facilitate easier implantation of implant 100 into a patient (not shown), but the same are not required to be. Furthermore, as shown in FIGS. 1-3, in one preferred embodiment, the body portion 104 of the PLIF implant 100 is capable of being expanded medial-laterally in vivo. More specifically, the body portion 104 can expand via a parallel expansion design which pushes the implant 100 open medial-laterally, as best shown in FIGS. 1-3; or via a pivot expansion design, which only expands on the anterior side of the implant 100, as best shown in FIGS. 4-6. Once expanded, flowable bone graft 134 can be inserted or pushed into the voids or plurality of graft windows 106, or other openings created by the expansion of the PLIF implant 100 to promote bone growth and/or osteo-integration, as best shown in FIGS. 2, 4, 5 and 6.

Additionally, in the parallel expansion design of the PLIF implant 100 as best shown in FIGS. 1 through 3, PLIF implant 100 may further comprise a partition 136 that is positioned within an opening 138 when PLIF implant 100 is in a closed or compressed position so as to retain the flowable graft material 134 in the center of the implant 100. In the pivot expansion design of implant 100, the expanding portions of body portion 104 of implant 100 pivot about a pin or pivot point 140 located at the rear of the implant 100, as best shown in FIGS. 4-6.

As best shown in FIGS. 1-5, opposing sides 114 are generally parallel and spaced apart from one another. One or more of opposing sides 114 may further comprise webbing, mesh or other honey-comb like structure 124 to promote bone growth within or around implant 100, as best shown in FIG. 3. More specifically, the webbed portion/honey-comb like structure 124 of opposing side walls 114 of implant 100 promotes osteo-integration and bone growth.

As best shown in FIGS. 1, 2 and 6, each of graft windows 106 is preferably a generally open portion in body portion 104 that extends from top 116 to bottom 118, and is positioned between opposing sides 114. Additional graft windows 106 may also be created when implant 100 is expanded, as best shown in FIGS. 2, 5 and 6. Graft windows 106 promote osteo-integration and bone growth within the disc space of the patient, and through implant device 100. Although the shape and dimensions of graft window (i.e., length, width, and height) are important design parameters for good performance, graft window 106 may be any shape or size that ensures optimal performance during use or that satisfies surgeon and/or patient need or preference.

Similarly, although the dimensions (i.e., length, width, and height) and the exact shape of implant 100 and its various components are important design parameters for good performance, implant 100 may be any shape or size that ensures optimal performance during use or that satisfies surgeon and/or patient need or preference. Notwithstanding, the overall height of implant 100, measured from the top 116 to the bottom 118, will typically be greater that its width, as measured between opposing sides 114, so that when implant 100 is inserted sideways in between two vertebral implants of a patient (not shown) and rotated approximately 90 degrees (clockwise or counterclockwise) about its centerline, implant 100 will become “taller” in the disc space and apply pressure of force against the adjacent vertebral implants, thereby causing greater distraction, which is desirable and results in better patient outcomes.

Typically, the PLIF and TLIF implants 100 of the present invention may be manufactured using additive manufacturing (AM) techniques, or using a combination of other molding or machining techniques (e.g., injection molding, machining, etc.) to produce the subject encoded implants. These additional techniques include, without limitation, material extrusion, vat photo polymerization, powder bed fusion, material jetting, binder jetting, sheet lamination and directed energy deposition. Typically, implant 100 is manufactured from titanium, specifically Ti 6 Al 4 V-ELI, but can be manufactured from any other suitable material as is known in the art for similar applications.

As shown in FIGS. 1-5, insertion tool 102 preferably comprises an elongated shaft 126, a handle and a rotatable rod (not shown). More specifically, shaft 126 is comprised of a proximal end (not shown), a distal end 130, and a longitudinal opening (not shown) therein extending from said proximal end to said distal end 130 for receipt of the rod.

Handle (not shown) is located at the proximal end of shaft 126 and is preferably comprised of a solid metal, such as stainless steel, though other materials may also be used provided that said materials are suitable for use in a sterile environment or surgical setting. Handle may have a scalloped gripping surface comprised of Radel® plastic or other suitable material to ensure steady handling of insertion tool 102, though any other durable handle known in the art could also be used. Handle further comprises an impactor cap (not shown) to aid in striking the handle during insertion of implant device 100, as explained more fully below, and a thumb wheel (not shown) mechanically attached to the rod so that rotation of thumb wheel will, in turn, also cause rod to rotate within shaft 126 about its centerline.

The distal end 130 of elongated shaft 126 (i.e., opposite handle and thumb wheel) is formed into a spaced apart grip member, comprising bifurcated and spaced apart shaft portions 128, as best shown in FIGS. 1-3. Shaft portions 128 form a gap 132 therebetween to securely retain the implant 100 during the implantation procedure. More specifically, shaft portions 128 may be inserted into spaced apart openings 122 between opposing sides 114, as explained more fully below.

As previously mentioned, the rod is positioned within an opening in the elongated shaft 126 and extends from its mechanical connection with thumb wheel in the direction of the distal end 130 of shaft 126, and into gap 132. Rod further comprises a threaded member (not shown) at its distal end for insertion into the opening 108, as more fully described below.

Having generally described a preferred embodiment of the PLIF implant device 100 and insertion tool 102 of the present invention, their function will now be generally described. A surgeon (not shown) desiring to install PLIF implant device 100 into a disc space (not shown) of a patient would attach implant 100 to insertion tool 102 by using the thumb wheel to rotate rod so that the threaded member is inserted into opening 108 and spaced apart shaft portions 128 are inserted into the spaced apart openings 122 between opposing sides 114. Because spaced apart openings 122 are internal to body portion 104, and positioned between and supported by both opposing sides 114 and sidewalls, implant 100 and insertion tool 102 are less prone to failure, bending or other damage than prior art PLIF implants and insertion tools, which typically grip the PLIF implant on its exterior surface thereby causing damage thereto during the implantation procedure.

Once the PLIF or TLIF implant 100 is properly installed on insertion tool 102, the surgeon may insert the PLIF of TLIF implant 100 into a disc space in a patient between two vertebras. Importantly, the chamfered surfaces 120 on front end 110 facilitate easier insertion of implant 100 in an oblique or straight posterior approach so that a single implant 100 can provide a patient with balanced support to the spinal column, and result in a better patient outcome. Once the implant 100 has been inserted sideways in between the two vertebras (not shown), implant 100 may be rotated approximately 90 degrees (clockwise or counterclockwise) about its centerline and, because implant 100 is generally taller (as measured from top 116 to bottom 118) than it is wide (as measured between opposing sides 114), implant 100 will become “taller” upon its rotation, and will apply pressure or force against the adjacent vertebral, thereby causing greater distraction, which is both desirable and tends to result in more positive patient outcomes.

Furthermore, the body portion 104 of the implant 100 may then be expanded medial-laterally (or pivotally, depending upon the particular design of implant 100 being utilized) in vivo. More specifically, the body portion 104 can be expanded via a parallel expansion design which pushes the implant 100 open medial-laterally, as best shown in FIGS. 1-3; or via a pivot expansion design which only expands in the anterior side of the implant 100, as best shown in FIGS. 4-5. Once expanded, flowable bone graft 134 can be pushed into the voids or graft windows 106 or other openings created by the expansion to promote bone growth and/or osteo-integration. Additionally, as explained more fully above, the presence of graft window 106 and mesh 124 on one or more of opposing sides 114 and/or sidewalls help to promote osteo-integration and bone growth within the disc space, and throughout implant device 100.

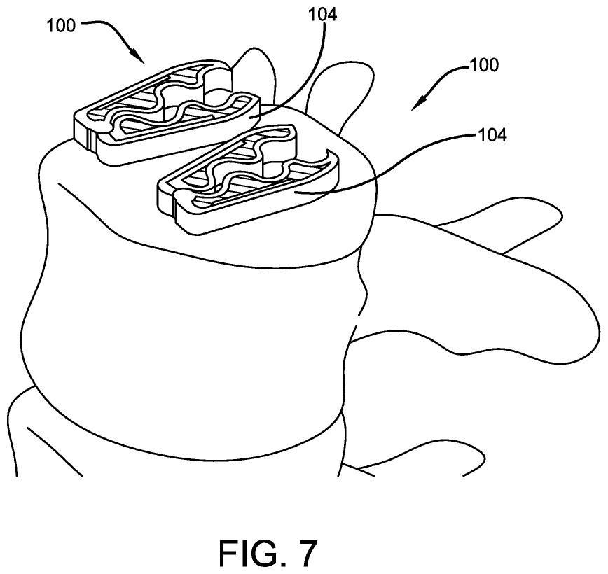

Furthermore, as best shown in FIGS. 6-7, a pair of implants 100 can be placed in the disc space of a patient, preferably in a straight or oblique posterior approach to achieve a larger overall footprint for greater stability of the construct and to achieve better patient outcomes. In use, one or two implants 100 can be used in the patient's disc space depending on the wants and/or needs of the surgeon performing the procedure and/or the particular patient requirements, and the implants 100 are preferably positioned in a generally side by side and spaced apart manner, as best shown in FIG. 6.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A posterior lumbar interbody fusion (PLIF) implant comprising:
a body portion having a first half and a second half; and
a graft window, wherein the PLIF implant is expandable.

2. The PLIF implant of claim 1, wherein the PLIF implant is expandable in a medial-lateral direction by repositioning the first half of the body portion away from the second half of the body portion.

3. The PLIF implant of claim 1 further comprising a pivot point, wherein the PLIF is expandable by pivoting the first half of the body portion about the pivot point and away from the second half of the body portion.

4. The PLIF implant of claim 1, wherein the PLIF implant is manufactured using additive manufacturing techniques.

5. The PLIF implant of claim 1, wherein the body portion is further comprised of a front end, a rear end, a pair of opposing sides, a top, and a bottom.

6. The PLIF implant of claim 5, wherein the rear end further comprises a continuous opening therein.

7. The PLIF implant of claim 5, wherein the front end is curved in shape and is comprised of one or more chamfered surfaces.

8. A posterior lumbar interbody fusion (PLIF) implant system comprising:
an expandable PLIF implant comprised of a body portion and a graft window, wherein the body portion is comprised of a pair of spaced apart openings; and
an insertion tool that secures to the pair of spaced apart openings to expand the expandable PLIF implant.

9. The PLIF implant system of claim 8, wherein the body portion further comprises a partition.

10. The PLIF implant system of claim 8, wherein the body portion further comprises a mesh surface for promoting osteo-integration.

11. The PUT implant system of claim 8, wherein the body portion is comprised of a first half and a second half.

12. The PLIF implant system of claim 11, wherein the expandable PLIF implant is expandable in a medial-lateral direction by repositioning the first half of the body portion away from the second half of the body portion.

13. The PLIF implant system of claim 11, wherein the expandable PLIF implant further comprises a pivot point, and further wherein the expandable PLIF implant is expandable by pivoting the first half of the body portion about the pivot point and away from the second half of the body portion.

14. The PLIF implant system of claim 8, wherein the expandable PLIF implant is manufactured using additive manufacturing techniques.

15. The PLIF implant system of claim 8, wherein the body portion is further comprised of a front end, a rear end, a pair of opposing sides, a top, and a bottom.

16. The PLIF implant system of claim 15, wherein the rear end further comprises a continuous opening therein.

17. The PLIF implant system of claim 15, wherein the front end is curved in shape and is comprised of one or more chamfered surfaces.

18. A spinal implant comprising:
an expandable body portion having a first half, a second half, a front end, a rear end, a pair of opposing sides, a top, and a bottom; and
a graft window, wherein the rear end further comprises a continuous opening and the front end is comprised of one or more chamfered surfaces.

19. The spinal implant of claim 18, wherein the expandable body portion is expandable in a medial-lateral direction by repositioning the first half of the body portion away from the second half of the body portion.

20. The spinal implant of claim 18 further comprising a pivot point, wherein the expandable body portion is expandable by pivoting the first half of the body portion about the pivot point and away from the second half of the body portion.
